(19) United States
(12) Patent Application Publication
(10) Pub. No.: US 2013/0218196 A1
(43) Pub. Date: Aug. 22, 2013

(54) AN ISCHEMIA AND REPERFUSION DEVICE

(71) Applicant: Michael Cheung, Parkville (AU)

(72) Inventor: Michael Cheung, Parkville (AU)

(73) Assignee: MURDOCH CHILDRENS RESEARCH INSTITUTE, PARKVILLE, VICTORIA (AU)

(21) Appl. No.: 13/813,874

(22) PCT Filed: Aug. 2, 2011
(86) PCT No.: PCT/AU2011/000977

§ 371 Date: Apr. 23, 2013

(30) Foreign Application Priority Data
Aug. 2, 2010 (AU) 2010903454

(51) Int. Cl. A61B 17/135

(52) U.S. Cl. CPC A61B 17/135
USPC 606/202

(57) ABSTRACT

A therapeutic ischemic and reperfusion device with an associated monitoring system for generally enhancing the vascular and metabolic environment and wellbeing of a subject. A method for the treatment and prophylaxis of various medical conditions including environmental induced oxidative stress using the therapeutic ischemic and reperfusion device and associated monitoring system is also contemplated herein. The method uses an inflatable cuff around the limb or torso of a subject operated by a controller configured to inflate and deflate the cuff. The monitoring system is used to monitor the physical and metabolic environments of the subject during and subsequent to the ischemia and reperfusion.

### FILING DATA

This application is associated with and claims priority from Australian Provisional Patent Application No. 2010903454, filed on 2 Aug. 2010, entitled “A medical device”, the entire contents of which, are incorporated herein by reference.

### FIELD

The present disclosure relates generally to a medical device and system including a therapeutic ischemic and reperfusion device with an associated monitoring system for generally enhancing the vascular and metabolic environment and wellbeing of a subject. A method for the treatment and prophylaxis of various medical conditions including environmental-induced oxidative stress using the therapeutic ischemic and reperfusion device and associated monitoring system is also contemplated herein.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Ischemic pre-conditioning is a term applied to an innate protective mechanism evolved to reduce ischemia-reperfusion injury (Murray et al, Circulation 74:1124-1136. 1986; Weber, Nature Medicine 16:760-762, 2010). Some studies have shown that temporary induced ischemia can be beneficial in coronary patients (Laskey and Beach, J. Am. Coll. Cardiol. 42:998-1003, 2003; Teoh et al. Cardiovasc. Res. 53:175-180, 2002). On the other hand, not all studies have been conclusive (Lessar et al., J. Am. coll. Cardiol. 42:175-180, 2003; Lindhardt et al, Heart 90:425-430, 2004). Remote ischemic pre-conditioning (rIPC), however, has been determined to have more clinical relevance (Przyklenk et al, Circulation 87:893-899, 1993). It has also been shown to be useful in children undergoing cardiopulmonary bypass to prevent myocardial ischemic reperfusion injury (Cheung et al, J. Am. Coll. Cardiol. 47:2277-2282, 2006). Prior to that work, Konstantinov et al, Physiol. Genomics. 19:143-150, 2004, proposed that rIPC could modify leukocyte inflammatory gene expression leading to a potential protective effect against ischemia-reperfusion injury. Das and Maulik, Cardiovascular Research 70:254-263, 2006, have proposed through gene expression array profiles, that changes in redox signaling is responsible for the generation of a pre-conditioning mediated “survival signal” from an ischemic-reperfusion-induced “death signal”. Sullivan et al, Brit. J. Surg. 96:381-390, 2009 have suggested that cyclical rIPC prevented lymphocyte-directed immune dysfunction while Linden et al, J. Throm. Haem. 4:2670-2677, 2006, suggested that rIPC attenuated platelet activation-aggregation.

There have been some attempts to develop rIPC devices. International Patent Application No. PCT/US2008/064792, for example, describes a device with an occluding member which operates by a remote controller. U.S. Pat. No. 7,717,855 also uses a controlled cuff device. However, the controller and monitoring aspects of these devices are designed to prevent prolonged occlusion by the operator. The devices are not designed for therapeutic use based on monitoring the vascular metabolic environment. Furthermore, since pressures greater than systolic pressure are used, this can cause substantial discomfort.

US Patent Publication No. 2010/0185220 describes the use of controlled ischemia such as a traumatic extravascular occlusions to treat a number of conditions. The ischemic and reperfusion cycles were generally in the 5-10 second to 60 second interval range.

US Patent Application No. 2010/0324429 describes a remote ischemic preconditioning and blood pressure monitoring device in the treatment or prevention of acute myocardial infarction. No data were provided, however, on its efficacy or is there any suggestion of a link between myocardial infarction treatment and metabolic wellbeing.

Whilst there is no doubt that rIPC has pleiotropic effects on various conditions, there is a need for an ability to develop a therapeutic ischemic and reperfusion device with an associated monitoring system which can be routinely used by individuals or carers to treat underlying pathological mechanisms associated with the metabolic environment as well as caused by environmental oxidative stress.

### SUMMARY

Enabled herein is a treatment device and its use to treat a subject to facilitate development and maintenance of an efficacious vascular and metabolic environment. The device is also useful in the treatment or prophylaxis of a subject exposed to environmental oxidative stress. One particular environmental stress is diet. A therapeutic ischemic and reperfusion device with an associated monitoring system is provided, predicated in part on a mechanism for inducing and monitoring ischemic and reperfusion conditioning by a controlled, releasable occlusion followed by reperfusion. This generally occurs in a cyclical manner of sequential occlusion followed by reperfusion. As taught herein, the therapeutic ischemic and reperfusion device with monitoring system is used to manipulate the vascular and metabolic environment in a subject and to promote general wellbeing and treat or prevent development of particular diseases or adverse conditions. It is also used to treat subjects exposed to oxidative stress by environmental factors such as high sugar and/or high fat diets.

Hence, an aspect enabled herein is a therapeutic ischemic and reperfusion device with an associated monitoring system comprising means to occlude fluid flow through a luminal vessel and induce a localized ischemia; means to release the occlusion to enable fluid flow through the luminal vessel: and means to monitor vascular and/or metabolic function at the location of the occlusion and/or remotely from the location of the occlusion. In an embodiment, the ischemia and reperfusion cycles occur under conditions and for a time sufficient to reduce insulin levels and/or reduce IL-6 levels. In another embodiment, the cycles improve myeloperoxidase, adiponectin and matrix metalloproteinase levels.

Reference to reducing insulin levels includes enhancing insulin sensitivity.

Also enabled herein is an environmental stress ameliorating device with an associated monitoring system comprising means to occlude fluid flow through a luminal vessel and induce a localized ischemia; means to release the occlusion to enable fluid flow through the luminal vessel; and means to monitor vascular and/or metabolic function at the location of the occlusion and/or remotely from the location of the occlusion.

Further taught herein is an ischemic and reperfusion device for subjects with abnormal levels of one or more of insulin, IL-6 and/or TNFα with an associated monitoring system comprising means to occlude fluid flow through a luminal vessel and induce a localized ischemia; means to release the occlusion to enable fluid flow through the luminal vessel; and means to monitor vascular and/or metabolic function at the location of the occlusion and/or remotely from the location of the occlusion.

In an embodiment, provided herein is a therapeutic ischemic and reperfusion device comprising:

an inflatable and deflatable pressure cuff or tourniquet adapted to fit around a limb or portion of a torso of a subject wherein upon inflation of the pressure cuff or tightening of the tourniquet, fluid flow through a luminal vessel in the limb or torso is occluded causing localized ischemia and, when deflated, enables the fluid to flow through the luminal vessel;

a controller operably connected to the pressure cuff or tourniquet configured to inflate the cuff or tighten the tourniquet to a selected pressure at least sufficient to occlude fluid flow through a luminal vessel and to deflate the cuff or release the tourniquet; and

a monitor configured to monitor luminal vessel and/or metabolic function at the site of the occlusion or remote to the site of occlusion to determine an efficacious vascular response. An efficacious vascular response improves levels of insulin and/or IL-6. An efficacious response is also associated with an improved level of one or more of IL-10, TNFα, adiponectin, myeloperoxidase and/or matrix metalloproteinase. By “levels” includes concentrations and velocities in fluid such as plasma, whole blood, scrum or lymph fluid.

The “selected” pressure is dependent on the disease or condition being treated or prevented.

For metabolic disease conditions such as Type II diabetes, obesity and certain inflammatory conditions, the selected pressure range is from the minimum pressure required to substantially occlude fluid flow through a luminal vessel to the systolic pressure or above. It also includes from the minimum pressure required to substantially occlude fluid flow through a luminal vessel up to but not including the systolic pressure. It also includes from the diastolic pressure up to but not including the systolic pressure.

The “certain inflammatory conditions” include inflammatory conditions of the respiratory system such as bronchitis, asthma and chronic obstructive pulmonary disease (COPD) and of the bowel such as inflammatory bowel disease, ulcerative colitis. Crohn's disease and pouchitis and inflammatory neuropathologies such as muscle dystrophies.

For metabolic diseases other than diabetes, obesity and the certain inflammatory conditions, the selected pressure is from the minimum pressure required to substantially occlude fluid flow through a luminal vessel up to but not including the systolic pressure.

Conditions contemplated in this aspect include chronic and acute inflammatory conditions. Examples of inflammatory disease conditions include acne, angina, arthritis, asthma, aspiration pneumonia disease, COPD, colitis, empyema, gastroenteritis, necrotizing enterocolitis, pelvic inflammatory disease, pharyngitis, pleurisy, chronic inflammatory demyelinating polyneuropathy, chronic inflammatory demyelinating polyradiculoncuropathy and muscle dystrophies.

Disease and conditions contemplated herein also included multiple sclerosis (MS), oligodendrocyte disease, acute disseminated encephalomyelitis, optic neuropathy (including neuromyelitis optic with transient autonomic disturbances). Devic's neuromyelitis optica, tropical spastic paraparesis, non-compressive myelopathies, concentric sclerosis, diffuse sclerosis acute hemorrhagic leukoencephalopathy, metachromatic leukodystrophy, leucoareois, acute discriminated encephalomyelitis, progressive multi focal leukoencephalopathy, multisystem entrophy, as well as any form of brain trauma resulting in white matter such as stroke or physical injury. All these disease conditions are encompassed by the terms “inflammatory neuropathological disease or condition”. “inflammatory neuropathology” and “neurodegenerative disease or condition”.

In an embodiment, the fluid is blood and the lumen is a blood vessel. By “monitoring metabolic function” includes monitoring luminal function.

The term “device” includes a treatment and prophylactic device, a medical device, a point-of-care device, a remote ischemic conditioning device, a home medical device, a metabolic facilitating device and other like terms. The “device” may also be referred to as an apparatus, unit, kit, implement, facility or other like term. In an embodiment, the device is a therapeutic ischemic and reperfusion device with an associated monitoring system. In another embodiment, the device is a home care unit or a point of care unit.

In an embodiment, the therapeutic ischemic and reperfusion device with associated monitoring system is useful for the treatment of diabetes, obesity and inflammatory conditions as well as improving exercise performance in humans and animals and treating or preventing chronic or acute diseases and/or conditions of the systemic and peripheral vasculature including connective tissue disease, cardiac dysfunction, stroke or brain hemorrhage as well as metabolic, endocrine and cardiovascular disorders or conditions which would benefit over time from improved circulatory including vascular endothelial function. Such conditions include those listed above. The device is also useful for modulating or otherwise ameliorating the effects of oxidative stress, such as caused by environmental stimuli. An example being diet. The improvement in metabolic environment induced by remote ischemic conditioning also has applications in farmaculture and in particular rearing lot animals such as cattle and pigs and in a protocol to prepare and maintain racing animals such as horses, dogs and camels.

It is proposed herein that the device leads to improved luminal vessel, such as blood vessel, function over time. This includes enhanced vascular function and in particular cardiovascular function over time. By improved luminal or blood vessel function includes increased vascular endothelial function over time. The device may be used alone or as part of a health program involving medicinal intervention and/or behavoral modification such as in relation to exercise, diet or stress management.

Hence, it is proposed herein that the device leads to an improved vascular and metabolic environment and vascular endothelial function including enhanced cardiovascular function over time. It also ameliorates the adverse consequences of the environment such as dietary oxidative stress. It is also useful for subjects with levels of one or more of insulin, IL-6, IL-10, TNFα, adiponectin, myeloperoxidase and/or matrix metalloproteinase which exacerbate or contribute to or which are otherwise associated with a disease or adverse condition.

The “levels” referred to above may be “abnormal” in which case the treatment regime promotes normalization of the levels. The levels may still be in a statistically normal range yet nevertheless contribute to a disease or condition. In this case the treatment modulates or improves the levels to a level which contributes to an amelioration of the disease or condition.

By improved metabolic environment function includes amelioration of symptoms associated with inflammatory responses and/or glucose and insulin resistance and/or sensitivity as well as reduction in obesity and/or exposure to environmentally induced oxidative stress.

The present disclosure teaches a therapeutic ischemic and reperfusion device with associated monitoring system for induction of remote ischemic pre-, post- or present-conditioning in a subject. For brevity, the effect is referred to herein as “remote ischemic conditioning”, which includes pre-conditioning, post-conditioning and present-conditioning.

An aspect enabled herein is a method for the treatment or prophylaxis of a metabolic condition in a subject selected from diabetes and obesity, the method comprising occluding luminal fluid flow in a vessel in the subject by the sequential application of circumferential pressure to a limb or torso on the subject containing the vessel for a time and under conditions sufficient to induce an ischemic condition, releasing the occlusion to induct reperfusion wherein the sequential ischemia and reperfusion results in a decrease in the levels of insulin compared to a subject not treated. As indicated above, another parameter to measure the effect of the treatment is to monitor any increase in insulin sensitivity.

Another aspect taught herein is a method for the treatment or prophylaxis of a metabolic condition in a subject having levels of one or more of IL-6, IL-10 and/or TNFα which exacerbate a disease or adverse condition, the method comprising occluding luminal fluid flow in a vessel in the subject by the sequential application of circumferential pressure to a limb or torso on the subject containing the vessel for a time and under conditions sufficient to induce an ischemic condition, releasing the occlusion to induce reperfusion wherein the sequential ischemia and reperfusion results in a modulation of these levels.

The modulated levels may mean an increase or decrease depending on the disease or condition.

Still another aspect enabled herein is a method for the treatment or prophylaxis of a metabolic condition in a subject having levels of one or more of adiponectin, myeloperoxidase and/or matrix metalloproteinase which exacerbate a disease or condition, the method comprising occluding luminal fluid flow in a vessel in the subject by the sequential application of circumferential pressure to a limb or torso on the subject containing the vessel for a time and under conditions sufficient to induce an ischemic condition, releasing the occlusion to induce reperfusion wherein the sequential ischemia and reperfusion results in a modulation of the levels.

Yet still another aspect enabled herein is a method for the treatment or prophylaxis of a subject exposed to environmentally-induced oxidative stress selected from diabetes and obesity, the method comprising occluding luminal fluid flow in a vessel in the subject by the sequential application of circumferential pressure to a limb or torso on the subject containing the vessel for a time and under conditions sufficient to induce an ischemic condition, releasing the occlusion to induce reperfusion wherein the sequential ischemia and reperfusion results ameliorating of the oxidative stress.

Even yet another aspect disclosed herein is a method for the treatment or prophylaxis of a disease or condition in a subject, the method comprising occluding luminal fluid flow in a vessel in the subject by the sequential application of circumferential pressure to a limb or torso on the subject containing the vessel, releasing the occlusion to include reperfusion wherein the sequential ischemia and reperfusion ameliorates symptoms of the disease or condition wherein the pressure applied is selected from the list consisting of:

(i) from the minimum pressure required to substantially occlude fluid flow through a luminal vessel to the systolic pressure or above for the treatment of Type II diabetes, obesity and selected inflammatory conditions; and

(ii) from the minimum pressure required to substantially occlude fluid flow through a luminal vessel up to but not including the systolic pressure for all other metabolic conditions.

As indicated above, selected inflammatory conditions include inflammatory conditions of the respiratory system such as bronchitis, asthma and COM, inflammatory conditions of the bowel such as inflammatory bowel disease, ulcerative colitis. Crohn's disease, and pouchitis, inflammatory conditions of the neurological system such as muscular dystrophies and inflammatory conditions of the joints.

In an embodiment, the physical and biochemical parameters are monitored. Physical parameters include detection of fluid flow, determination of pulse rate, determination of heart beat rate, determination of changes in temperature, determination of responses by capillaries, an assessment of flow mediated dilatation, measurements of blood flow and pulse wave velocity, pulse wave analysis, bio-impedance analysis, heart rate variability and measurement of 24 hour blood pressure. As well as respirator indicators of lung function such as standard spirometry and pulmonary function techniques.

Biochemical parameters include detection of an anti-inflammatory, inflammatory and/or pro-inflammatory cytokine, detection of a platelet aggregation factor, detection of oxygen levels, detection of carbon dioxide levels and detection of hemoglobin, lactate, pH. ATP, ADP, AMP, adenosine, redox voltage, erythropoietin and bradykinin levels. Biochemical parameters also include insulin. IL-6, IL-10, TNFα, adiponectin, myeloperoxidase and/or matrix metalloproteinase.

Monitoring may be at the site of the occlusion or remote from the site of the occlusion.

Still another aspect taught therein is a method for the treatment or prophylaxis of a subject exposed to or who may be exposed to environmentally-induced oxidative stress, the method comprising occluding luminal fluid flow in a vessel in the subject by the sequential application of circumferential pressure to a limb or torso on the subject containing the vessel for a time and under conditions sufficient to induce an ischemic condition, releasing the occlusion to induce reperfusion wherein the sequential ischemia and reperfusion results in amelioration of the oxidative stress levels.

This embodiment is particularly useful in treating diet-induced oxidative stress. By “normalization of oxidative stress levels” includes normalization of the levels of insulin, IL-6, IL-10 and/or TNFα.

A method is also provided for the treatment or prophylaxis of a metabolic condition in a subject selected from diabetes and obesity, the method comprising applying an inflatable and deflatable pressure cuff or tourniquet adapted to fit around a limb or portion of a torso of the subject wherein upon inflation of the pressure cuff or tightening of the tourniquet, fluid flow through a luminal vessel in the limb or torso is occluded causing localized ischemia and, when deflated, enables fluid to flow through the luminal vessel and subjecting the pressure cuff or tourniquet to multiple cycles of inflation and deflation or tightening and releasing operated by a controller operably connected to the pressure cuff or tourniquet wherein the pressure applied is between the pressure required to substantially occlude fluid flow through a luminal vessel and up to but not including the systolic pressure, the multiple cycles of inflation and deflation or tightening and releasing being for a time and under conditions for an efficacious vascular response compared to a subject which has not undergone the treatment. In an embodiment, the fluid is blood and the luminal vessel is a blood vessel.

Conveniently, a subject can be monitored during or after the treatment for a normalization of one or more markers selected from insulin, IL-6, IL-10 and/or TNFα, as well as adiponectin, myeloperoxidase and/or matrix metalloproteinase.

The present disclosure further teaches a therapeutic ischemic and reperfusion device with an associated monitoring system comprising an occlusion member adapted to releaseably restrict luminal fluid flow following circumferential pressure applied to an extremity in which a lumen is located; and a monitoring member associated with the occlusion member which measures physical or biochemical parameters of luminal function and/or metabolic environment at the site of the occlusion, proximal to the site of the occlusion or at a location remote or distal to the occlusion. In an embodiment, the fluid is blood and the lumen is a blood vessel.

The medical device enabled herein is largely non-invasive in the sense that the occlusion is induced by releasable circumferential pressure. However, the monitoring device may include a fluid testing component which may require a fluid sample being taken such as to determine blood glucose levels or other biochemical markers.

Business models to monitor metabolic wellbeing in subjects are also taught herein.

In an embodiment, the device is installed for public use at food outlets which supply high fat and/or high sugar content foods. The use may be at a cost to the individual user or supply may be provided at a cost to the food supplier. The device may also be packaged for sale, such as in kit form with instructions for use.

Abbreviations used herein are defined in Table 1.

TABLE 1
Abbreviations

| Abbreviation | Definition |
|---|---|
| AUC | Area under the curve |
| HOMA | Homeostatic model of assessment |
| HOMA-IR | Homeostatic model of assessment for insulin resistance |
| IL-10 | Interleukin-10 |
| IL-6 | Interleukin-6 |
| IR | Insulin resistance |
| RHI | Reactive hyperemic index |
| rIPC | Remote ischemic preconditioning |
| TNFα | Tumor necrosis factor alpha |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a graphical representation showing the significant change (p<0.003) in the reactive hyperemic index (RHI) as a percentage of pre-meal values (time 0) and at 1 and 2 hours after a high-fat, high-glucose meal. Initial response (baseline; squares) is a reduction in vessel function and post-chronic rIPC (daily treatment) values (post-rIPC triangles) vascular function is preserved. It is proposed that RHI is a measure of vascular function.

FIGS. 2A and B is a graphical representation showing levels of the cytokine, IL-6 in subjects (n=16) receiving treatment with rIPC vs controls (baseline) at pre-meal (time 0) and then at 1 and 2 hours after eating a high fat, high glucose meal (A). There was a significant difference in the AUC (B) of the two groups (p=0.01) with lower levels in the treatment group.

FIGS. 3A through C are graphical representations showing controlled serum glucose levels (A) in normal healthy males (n=18) after ingestion of a high-fat, high-glucose meal both at baseline and after a week of daily rIPC. Lower levels of insulin (B) were released after the week of rIPC indicting improved sensitivity as shown by the lower area under the curve (AUC) for homeostatic model assessment for insulin resistance (HOMA-IR) HOMA-IR (C) after consumption of the standard high fat and high glucose meal at baseline prior to and following a week of daily preconditioning by inflating the cuff on the upper arm greater than systolic blood pressure for 3 cycles of 5 minutes of ischaemia followed by cuff release and 5 minutes of reperfusion.

FIGS. 4A through C are graphical representations showing glucose (A), insulin (B) and homeostatic model assessment (HOMA) (C) using the diastolic occlusion protocol taught herein. The cuff being placed around the upper limb and inflating to 20 mmHg above diastolic blood pressure. Other than the difference in the magnitude of the cuff inflation pressure, the same protocol of daily sessions of 3 cycles of inflation for 5 minutes followed by release of the cuff for 5 minutes was followed for 1 week.

FIG. 5 is a graphical representation showing levels of myeloperoxidase (MPO) in the body are an indicator of the level of oxidative stress and the activation of white blood cells in response to injury or danger.

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method step or group of elements or integers or method steps.

As used in the subject specification, the singular forms “a”, “an” and “the” include plural aspects unless the context clearly dictates otherwise. Thus, for example, reference to “a parameter” includes a single parameter, as well as two or more parameters; reference to “an occlusion” includes a single occlusion, as well as two or more occlusions; reference to “the disclosure” includes single or multiple aspects taught and enabled by the disclosure; and so forth.

The present disclosure teaches a therapeutic ischemic and reperfusion device with associated monitoring system and its use to facilitate an efficacious vascular endothelial and metabolic environment and over all associated metabolic wellbeing in a subject. The device is predicated in part on a mechanism for inducing and monitoring remote ischemic conditioning (rIPC) by controlled, releasable occlusions followed by reperfusions. Generally, the present disclosure provides a cyclical protocol of ischemia and reperfusion to induce remote ischemic conditioning in order to manipulate the vascular endothelial and metabolic environment. The latter includes reducing levels of inflammatory and pro-inflammatory cytokines, reducing inflammatory markers, enhancing insulin sensitivity and glucose tolerance, reducing the incidence of diabetes or risk of developing same and reducing obesity. The device is also useful for ameliorating the effects of environmentally-induced oxidative stress such as diet-induced oxidative stress. The device can also be used in the treatment of inflammatory based diseases of the respiratory system, bowel and gut, neurological system and joints.

Hence, an aspect enabled herein is a therapeutic ischemic and reperfusion device with an associated monitoring system, the device comprising:

(i) means to occlude fluid flow through a luminal vessel and induce a localized ischemia;

(ii) means to release the occlusion to enable fluid flow through the luminal vessel; and

(iii) means to monitor metabolic function at the location of the occlusion and/or remotely from the location of the occlusion.

In an embodiment, provided herein is a therapeutic ischemic and reperfusion device comprising:

an inflatable and deflatable pressure cuff or tourniquet adapted to fit around a limb or portion of a torso of a subject wherein upon inflation of the pressure cuff or tightening of the tourniquet, fluid flow through a luminal vessel in the limb or torso is occluded causing localized ischemia and, when deflated, enables the fluid to flow through the luminal vessel;

a controller operably connected to the pressure cuff or tourniquet configured to inflate the cuff or tighten the tourniquet to a selected pressure and to deflate the eta or release the tourniquet; and

a monitor configured to monitor luminal vessel and/or metabolic function at the site of the occlusion or remote to the site of occlusion to determine an efficacious vascular response. In an embodiment, an efficacious vascular response is a reduction in levels of insulin and/or IL-6. In another embodiment, an efficacious vascular response is monitored by modulation of levels of one or more of insulin, IL-6, IL-10, TNFα, adiponectin, myeloperoxidase and/or matrix metalloproteinase. In many circumstances, modulation will be a normalization of levels such as reducing the levels of these biomarkers to normal levels. However, certain disease conditions result from reduced levels of one or more of the these biomarkers, hence, normalization involves an increase in levels to normal levels. The up or down regulation of levels is encompassed by the term “modulation”. By “normal” is generally meant a baseline level in a healthy subject without any disease condition associated with abnormal levels of one or more of insulin, IL-6, IL-10, TNFα, adiponectin, myeloperoxidase and/or matrix metalloproteinase.

Hence, the “levels” referred to above may be “abnormal” in which case the treatment regime promotes normalization of the levels. The levels may still be in a statistically normal range yet nevertheless contribute to a disease or condition. In this case the treatment modulates or improves the levels to a level which contributes to an amelioration of the disease or condition.

Conveniently, an efficacious vascular response can also be determined using homeostatic model assessment (HOMA) which was first described by Matthews et al., Diabetologia 28(7):412-419, 1985. The HOMA model for insulin resistance (IR) is referred to as HOMA-IR.

The “selected” pressure is dependent on the disease or condition being treated or prevented.

For metabolic disease conditions such as Type II diabetes, obesity and certain inflammatory conditions, the selected pressure range is from the minimum pressure required to substantially occlude fluid flow through a luminal vessel to the systolic pressure or above. It also includes from the minimum pressure required to substantially occlude fluid flow through a luminal vessel up to but not including the systolic pressure. It also includes from the diastolic pressure up to but not including the systolic pressure.

Certain inflammatory conditions include inflammatory conditions of the respiratory system such as bronchitis, asthma and chronic obstructive pulmonary disease (COPD) and of the bowel such as inflammatory bowel disease, ulcerative colitis, Crohn's disease and pouchitis and inflammatory neuropathologies such as muscle dystrophies.

For metabolic diseases other than diabetes, obesity and certain types of inflammatory conditions, the selected pressure is from the minimum pressure required to substantially occlude fluid flow through a luminal vessel up to but not including the systolic pressure.

Conditions contemplated in this aspect include chronic and acute inflammatory conditions. Examples of inflammatory disease conditions include acne, angina, arthritis, asthma, aspiration pneumonia disease, COPD, colitis, empyema, gastroenteritis, necrotizing enterocolitis, pelvic inflammatory disease, pharyngitis, pleurisy, chronic inflammatory demyelinating polyneuropathy, chronic inflammatory demyelinating polyradiculoneuropathy and muscle dystrophies.

Disease and conditions contemplated herein also included multiple sclerosis (MS), oligodendrocyte disease, acute disseminated encephalomyelitis, optic neuropathy (including neuromyelitis optic with transient autonomic disturbances). Devic's neuromyelitis optica, tropical spastic paraparesis, non-compressive myelopathies, concentric sclerosis, diffuse sclerosis acute hemorrhagic leukoencephalopathy, metachromatic leukodystrophy, leucoareois, acute discriminated encephalomyelitis, progressive multifocal leukoencephalopathy, multisystem entrophy, as well as any form of brain trauma resulting in white matter such as stroke or physical injury. All these disease conditions are encompassed by the terms “inflammatory neuropathological disease or condition”. “inflammatory neuropathology” and “neurodegenerative disease or condition”.

In an embodiment, the fluid is blood and the lumen is a blood vessel.

Accordingly, another aspect of the present disclosure is directed to a therapeutic ischemic and reperfusion device with associated monitoring system, the device comprising:

(i) means to occlude blood fluid through a blood vessel and induce a localized ischemia;

(ii) means to release the occlusion to enable blood fluid through the blood vessel; and

(iii) means to monitor metabolic function at the location of the occlusion and/or remotely from the location of the occlusion.

In an embodiment, provided herein is a therapeutic ischemic and reperfusion device comprising:

an inflatable and deflatable pressure cuff or tourniquet adapted to fit around a limb or portion of a torso of a subject wherein upon inflation of the pressure cuff or tightening of the tourniquet, blood flow through a blood vessel in the limb or torso is occluded causing localized ischemia and, when deflated, enables the blood to flow through the blood vessel;

a controller operably connected to the pressure cuff or tourniquet configured to inflate the cuff or tighten the tourniquet to a selected pressure and, to deflate the cuff or release the tourniquet; and

a monitor configured to monitor blood vessel and/or metabolic function at the site of the occlusion or remote to the site of occlusion to determine an efficacious vascular response. An efficacious vascular response is as indicated above such as a modulation of levels of one or more of insulin, IL-6, IL-10, INFα, adiponectin, myeloperoxidase and/or matrix metalloproteinase. These levels may be in any suitable fluid such as whole blood, plasma, serum, lymph fluid, tissue extract fluid, urine, respiratory fluid and the like.

As above, the selected pressure is selected from:

(i) the minimum pressure required to substantially occlude fluid flow through a luminal vessel up to systolic pressure or above; and

(ii) the minimum pressure required to substantially occlude fluid flow through a luminal vessel up to but not including the systolic pressure.

The term “device” includes a treatment and prophylactic device, a medical device, a point-of-care device, a remote ischemic conditioning device, a home medical device, a metabolic facilitating device and other like terms. The “device” may also be referred to as an apparatus, unit, kit, implement, facility or other like term. In one embodiment, the device is a remote ischemic conditioning unit or more particularly a therapeutic ischemic and reperfusion device with associated monitoring system. The device may also be in component form requiring some assembly prior to use.

Another aspect enabled herein provides a therapeutic ischemic and reperfusion device with associated monitoring system to induce remote ischemic conditioning and enhance the metabolic environment in a subject, the device comprising:

(i) means to occlude fluid through a luminal and induce a localized ischemia;

(ii) means to release the occlusion to enable fluid through the luminal; and

(iii) means to monitor vascular function at the location of the occlusion and/or remotely from the location of the occlusion.

Taught herein is a therapeutic ischemic and reperfusion device with associated monitoring system to induce remote ischemic conditioning and enhance the metabolic environment in a subject, the device comprising:

(i) means to occlude blood flow through a blood vessel and induce a localized ischemia;

(ii) means to release the occlusion to enable blood flow through the blood vessel; and

(iii) means to monitor vascular function at the location of the occlusion and/or remotely from the location of the occlusion.

Reference to the “metabolic environment” and “metabolic wellbeing” includes the vascular endothelial and endocrine and cardiovascular environments and encompasses the underlying vascular, peripheral, hematological, physiological, cardiovascular, neurological and organ function required to maintain health and wellbeing in a subject and to prevent or reduce the incidence of a range of metabolic-like diseases and/or metabolic syndromes. In one aspect, the metabolic environment includes vascular endothelial function and overall cardiovascular function. Diseases and conditions associated with the metabolic environment include diseases and conditions of the systemic and peripheral vasculature, especially those conditions which have an inflammatory component as part of, or which exacerbates, a pathological condition. Examples include diabetes (in particular Type 2 diabetes), connective tissue and joint disease, cardiac dysfunction, obesity, respiratory disease, insulin sensitivity or resistance, stroke and brain hemorrhage. Other conditions adversely affecting the metabolic environment is environmentally-induced oxidative stress such as diet-induced oxidative stress. This can be observed in subjects who consume high sugar and/or high fat content foods.

The present device and method has application in the treatment or prophylaxis of any disease or adverse condition. Generally, for diabetes, obesity and inflammatory conditions of the respiratory system, gastrointestinal system, joints and neurological system, the device is used at an occluding pressure of from the minimum pressure required to occlude fluid flow through a lumen to the systolic pressure or above. For all other conditions such as chronic and acute inflammatory conditions. Examples of inflammatory disease conditions include acne, angina, arthritis, asthma, aspiration pneumonia disease. COPD, colitis, empyema, gastroenteritis, necrotizing enterocolitis, pelvic inflammatory disease, pharyngitis, pleurisy, chronic inflammatory demyelinating polyneuropathy, chronic inflammatory demyelinating polyradiculoneuropathy and muscle dystrophies but including diabetes, obesity and inflammatory conditions of the respiratory system, gastrointestinal system, joints and neurological system, the pressure is from the minimum pressure required to occlude fluid flow through a lumen up to but including the systolic pressure.

Taught herein is therapeutic intervention which over time ameliorates vascular endothelial and metabolic conditions associated with diabetes, inflammation, cardiovascular disease, obesity, glucose intolerance and insulin resistance as well as which ameliorates the adverse effects of environmentally-induced oxidative stress. The device can also be used for a range of other conditions including inflammatory bowel disease. Crohn's disease, ulcerative colitis and pouchitis, respiratory disease such as bronchitis, asthma and COPD, joint disease such as arthritis and neurological diseases such as muscular dystrophies.

Further enabled is a therapeutic ischemic and reperfusion device to treat a metabolic condition selected from diabetes and obesity, the device comprising:

an inflatable and deflatable pressure cuff or tourniquet adapted to fit around a limb or portion of a torso of a subject wherein upon inflation of the pressure cuff or tightening of the tourniquet, blood flow through a blood vessel in the limb or torso is occluded causing localized ischemia and, when deflated, enables the blood to flow through the blood vessel;

a controller operably connected to the pressure cuff or tourniquet configured to inflate the cuff or tighten the tourniquet to a selected pressure and to deflate the cuff or release the tourniquet; and

a monitor configured to monitor blood vessel and/or metabolic function at the site of the occlusion or remote to the site of occlusion to determine an efficacious vascular response. In an embodiment, the metabolic condition is an inflammatory response.

The device is particularly useful, therefore, for the treatment of diabetes, obesity and inflammation including the treatment of an inflammatory response. An aspect herein teaches an increase in insulin sensitivity and a decrease in IL-6 as well as modulation of levels of one or more of IL-10, TNFα, adiponectin, myeloperoxidase and/or matrix metalloproteinase to levels which ameliorate by inflammatory response.

The therapeutic ischemic and reperfusion device with associated monitoring system is also proposed to assist in facilitating exertion performance of human athletes including endurance athletes, long distance runners, cyclers and swimmers and sprinters as well as non-human racing animals such as racehorses, race camels, greyhounds and working dogs. In this regard, the device may be used to screen for subjects who would be expected to out perform other subjects based on their level of vascular wellbeing. The term “subject” as applied to a recipient of remote ischemic conditioning includes, therefore, a human and non-human animal. Non-human animals include, apart from racing animals listed above, non-human primates, farm animals such as cattle, sheep, pigs and goats as well as domestic companion animals such as cats. The subject may be selected such as on the basis of a physical profile of age, weight and/or other parameters, a biochemical profile of levels of particular markers or on a responsive profile of being able to respond within a certain time selected from 24 hours to 3 weeks including 1, 2 or 3 weeks.

In an embodiment, the medical device is useful for improving exercise performance and treating or preventing chronic or acute diseases and/or conditions of the systemic and peripheral vasculature including those having an inflammatory component underlying a pathological or pathogenetic mechanism such as diabetes, connective tissue disease, cardiac dysfunction, obesity, insulin sensitivity or resistance, stroke or brain hemorrhage. The improvement in metabolic environment induced by remote ischemic conditioning also has applications in farmaculture and in particular rearing lot animals such as cattle and pigs. The device may be used alone or in combination with medicinal intervention and/or modified behavoral protocols such as diet and exercise.

The occlusion of fluid flow such as blood flow is through any vessel where pressure can be applied, effectively around the vessel or around tissue surrounding the vessel. Hence, the occlusion may be induced by applying pressure around a limb or other tissue including the torso of a subject. By “limb” includes an arm or leg or parts thereof such as a hand, finger, foot or toe. Reference to “other tissue” includes a neck, stomach, abdomen and car. The general term “torso” is used when not referring to a limb. The term “extremity” may also be used to describe an area of a subject's body which may be subjected to circumferential pressure to restrict fluid flow through a luminal vessel, and in particular, blood flow through a blood vessel. The term “appendage” also includes an extremity as does “torso”. Release of the occlusion results in reperfusion.

The occlusion may occur by any convenient means, notably by a pressure cuff or tourniquet where pressure is applied around the circumference of the vessel or the tissue containing the vessel. The pressure cuff or tourniquet applies a constricting or tightening force around the vessel resulting in construction of fluid flow through the vessel. Whilst pressure cuffs and tourniquets are the most convenient means of occluding fluid flow through a vessel, there are other mechanisms which could be applied such as through use of releasable ratchet, ringed or jawed clamps. All such mechanisms for releasable and temporary occlusion of fluid flow are contemplated herein. In this regard, therefore, the device requires a means to occlude fluid flow as well as a means to release the occlusion and induce reperfusion. Conveniently, when a pressure cuff is used, the means to release the occlusion includes releasing the pressure within the cuff. Similarly, if a tourniquet is used or other mechanism, the constrictive pressure is released by mechanical or automatic action.

Also taught herein is the temporary occlusion of fluid flow through a luminal vessel. By “fluid” is meant to include blood, lymph fluid, urine or tissue fluid. Generally, however, the fluid is blood. The luminal vessel includes an artery, vein, capillary and lymph vessel or other tissue structure which transports or carries blood or fluid material within the body. For purposes of the present disclosure, the luminal vessel is generally a blood vessel located within an extremity that can be readily subject to circumferential pressure to occlude fluid including blood flow.

Although not intending to limit the present disclosure to any one theory or mode of action, it is proposed that temporary (i.e. releasable) occlusion of luminal fluid flow, generally blood fluid, results in at least partial ischemia leading to ischemic conditioning which in turn is useful for the therapeutic intervention of conditions such as diabetes, cardiovascular disease, inflammation, obesity, glucose intolerance and insulin resistance. It is proposed that the therapeutic ischemic and reperfusion device with associated monitoring system facilitates remote ischemic conditioning. The term “remote ischemic conditioning” includes remote pre-conditioning as well as post-conditioning and encompasses remote ischemic and reperfusion treatment. By “ischemic” is meant a lowering of baseline fluid flow within a vessel from one point to another. Partial occlusion of fluid flow can lead to a state of hypoxia resulting from a lowering of partial oxygen (pO₂) levels. Upon release of the temporary occlusion, there is an immediate increase in fluid now resulting in reactive hyperemia which is defined as an increase in fluid flow from one point to another following release of the occlusion. It is proposed herein that the temporary occlusion followed by release or reperfusion for a defined time period results in increased reactive hyperemic or an increased hyperemic index over time. This includes improved luminal vessel such as blood vessel function over time.

The term “occlusion” means the partial or total ceasing or reduction in luminal fluid flow, including through an artery, vein, capillary or lymph vessel. Terms such as “extravascular occlusion” or “extraluminal occlusion” may be used to describe circumferential pressure applied to an extremity carrying the lumen to be occluded to result in the luminal occlusion of fluid flow. As above, the selected pressure is selected from:

(i) the minimum pressure required to substantially occlude fluid flow through a luminal vessel up to systolic pressure or above; and

(ii) the minimum pressure required to substantially occlude fluid flow through a luminal vessel up to but not including the systolic pressure.

In an embodiment, the pressure is from between the diastolic pressure up to but not including the systolic pressure. In an embodiment, the pressure is from between diastolic and up to but not including systolic. The expression “systolic pressure or above” means from about 1 mmHg to 100 mmHg above the systolic pressure. This includes 1, 2, 3, 4, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100 mmHg. Generally, the diastolic pressure is the minimum systemic arterial pressure which will vary for each subject but may be in the range 60-120 mmHg. The systolic pressure will also vary between subjects and is the maximum systemic arterial pressure which may be from 90-180 mmHg. A person of skill in the art will readily be able to determine a subject's diastolic and systolic pressures.

Hence, another aspect taught herein is a device for therapeutically enhancing a vascular endothelial metabolic environment in a subject, the device comprising:

(i) means to occlude fluid through a luminal and induce a localized ischemia;

(ii) means to release the occlusion to enable fluid through the luminal; and

(iii) means to monitor vascular function at the location of the occlusion and/or remotely from the location of the occlusion;

wherein in use, the device leads to improved luminal function over time.

Particularly, the present disclosure teaches a device for therapeutically enhancing a metabolic environment in a subject, the device comprising:

(i) means to occlude blood flow through a blood vessel and induce a localized ischemia;

(ii) means to release the occlusion to enable blood flow through the blood vessel; and

(iii) means to monitor vascular function at the location of the occlusion and/or remotely from the location of the occlusion;

wherein in use, the device leads to improved blood vessel function over time.

By “improved blood vessel function” includes enhanced vascular and in particular cardiovascular function over time.

In another aspect, the present disclosure enables a device for therapeutically enhancing a metabolic environment in a subject, the device comprising:

(i) means to occlude blood flow through a blood vessel and induce a localized ischemia;

(ii) means to release the occlusion to enable blood flow through the blood vessel; and

(iii) means to monitor vascular function at the location of the occlusion and/or remotely from the location of the occlusion;

wherein in use, the device leads to a reduction in pro-inflammatory cytokines, inflammatory cytokines and/or inflammatory markers over time.

In still another aspect enabled herein is a device for therapeutically enhancing a metabolic environment in a subject, the device comprising:

(i) means to occlude blood flow through a blood vessel and induce a localized ischemia;

(ii) means to release the occlusion to enable blood flow through the blood vessel; and

(iii) means to monitor vascular function at the location of the occlusion and/or remotely from the location of the occlusion;

wherein in use, the device leads to a reduction in glucose intolerance and/or sensitivity and/or insulin resistance.

Even yet another aspect taught herein, is a device for therapeutically enhancing a metabolic environment in a subject, the device comprising:

(i) means to occlude blood flow through a blood vessel and induce a localized ischemia;

(ii) means to release the occlusion to enable blood flow through the blood vessel; and

(iii) means to monitor vascular function at the location of the occlusion and/or remotely from the location of the occlusion;

wherein in use, the device ameliorates the symptoms of diabetes, obesity and/or cardiovascular diseases.

As indicated above, “improved luminal vessel function” and “improved blood vessel function” includes increased vascular and more particularly cardiovascular function over time.

Another aspect taught herein is a therapeutic device comprising:

(i) means to occlude fluid through a luminal and induce a localized ischemia;

(ii) means to release the occlusion to enable fluid through the luminal; and

(iii) means to monitor vascular function at the location of the occlusion and/or remotely from the location of the occlusion;

wherein in use, the device leads to increased vascular function over time.

The present disclosure teaches a therapeutic device comprising:

(i) means to occlude blood flow through a blood vessel and induce a localized ischemia;

(ii) means to release the occlusion to enable blood flow through the blood vessel; and

(iii) means to monitor vascular function at the location of the occlusion and/or remotely from the location of the occlusion;

wherein in use, the device leads to increased cardiovascular function over time.

Increased cardiovascular function encompasses increased reactive hyperemia.

The present disclosure extends to the induction of remote ischemic pre-, post- or present-conditioning in a subject. For brevity, the ischemia is referred to herein as “remote ischemic conditioning”, which includes pre-conditioning, post-conditioning and present-conditioning. Also as indicated above, the present disclosure extends to the occlusion of any fluid vessel.

Another aspect taught herein is a therapeutic ischemic and reperfusion device with associated monitoring system to induce remote ischemic conditioning device comprising:

(i) an occlusion member adapted to releaseably restrict luminal fluid flow following circumferential pressure applied to an extremity in which a lumen is located; and

(ii) a monitoring member associated with the occlusion member which measures physical or biochemical parameters of luminal function and/or metabolic environment at the site of the occlusion, proximal to the site of the occlusion or at a location remote or distal to the occlusion.

Particularly, disclosed is a therapeutic ischemic and reperfusion device with associated monitoring system to induce remote ischemic conditioning device comprising:

(i) an occlusion member adapted to releaseably restrict blood fluid flow following circumferential pressure applied to an extremity in which a blood vessel is located; and

(ii) a monitoring member associated with the occlusion member which measures physical or biochemical parameters of blood vessel function and/or metabolic environment at the site of the occlusion, proximal to the site of the occlusion or at a location remote or distal to the occlusion.

It is proposed herein that the therapeutic device herein results in improved metabolic function in the subject as determined by inter alia improved cardiovascular health, improved cardiovascular performance, improved vessel or luminal function and/or improved reactive hyperemia. As indicated above, reference to “metabolic function” includes endocrine function and cardiovascular function as well as the respiratory system such as for athletes and racing or working, animals. Also as indicated above, the device may also be used in conjunction with medicinal intervention and/or with behavoral modification in relation to diet, exercise and managing stress.

The monitoring means is a monitoring member or device which enables determination of physical and/or biochemical parameters at or close to the site of the occlusion and/or remote or distal to the site of the occlusion. For example, the occlusion may be induced on one arm or leg and the effects monitored on the other arm or leg. Physical parameters which can be detected include any associated with luminal function such as fluid (e.g. blood) flow, determination of pulse rate, determination of heart beat rate, determination of changes in temperature (regional, local or distal body temperatures), determination of responses by capillaries, assessment of flow mediated dilatation, and measurement of fluid flow, pulse wave velocity, pulse wave analysis, bio-impedance analysis, heart rate variability and measurement of 24 hour blood pressure. These parameters are conveniently determined by, for example, strain gauge plethysmography, fluid flow detection, pulse detection, temperature changes, peripheral plethysmography, saturation monitoring and vessel rarefaction. Respiratory parameters may also be measured such as to measure lung function and capacity.

Biochemical parameters which may be detected or measured include inter alia fluid (e.g. blood) glucose levels, insulin levels, insulin sensitivity or resistance, anti-inflammatory, inflammatory and/or pro-inflammatory cytokines, platelet aggregation factors, oxygen levels, carbon dioxide levels, hemoglobin levels, lactate, pH, ATP, ADP, AMP, adenosine, redox voltage, erythropoietin and/or bradykinin, as well as specifically one or more of insulin, IL-6, IL-10, TNFα, adiponectin, myeloperoxidase and/or matrix metalloproteinase.

In an embodiment, the physical and biochemical parameters are determined using plethysmography or other convenient apparatus or system.

Another aspect taught herein is a therapeutic ischemic and reperfusion device with associated monitoring system, the device comprising:

(i) an occlusion member adapted to releaseably restrict luminal fluid flow following circumferential pressure applied to an extremity of a subject's body in which a lumen is located; and

(ii) a plethysmographic monitoring member or other detection or monitoring member including a lung function monitor associated with the occlusion member which measures changes in volume of fluid and/or air within a subject at or remote from the site of the occlusion.

The present disclosure is also directed to therapeutic ischemic and reperfusion device with associated monitoring system, the device comprising:

(i) an occlusion member adapted to releasably restrict blood fluid flow Following circumferential pressure applied to an extremity of a subject's body in which a blood vessel is located; and

(ii) a plethysmographic monitoring member or other detection or monitoring monitor including a lung function monitor such as using standard spirometry pulmonary function techniques associated with the occlusion member which measures changes in volume of fluid and/or air within a subject at or remote from the site of the occlusion.

Taught herein is the use of atraumatic extraluminal occlusions to induce ischemic conditioning (which includes pre-, post- and present-conditioning). The occluding member, as indicated above, enables releasable pressure to be applied to an extremity such as an arm, leg, finger, toe, neck, stomach or lower abdomen for a pre-determined or controlled time. The circumferential pressure being determined to restrict fluid movement through one or more luminal vessels. The amount of pressure applied or required to restrict fluid movement and the length of time of occlusion will vary depending on the condition being treated or prevented and age, sex, weight and other parameter of the subject being treated. In an embodiment, the medical device is designed for home use and is, hence, portable. In another embodiment, the apparatus is used at a point-of-care Facility which enables its connection to more elaborate monitoring devices.

As indicated above, the subject may be a human or non-human animal. Human subjects include those being treated for conditions such as inflammation, arthritis, diabetes, obesity, cardiovascular disease or other conditions of the systemic and peripheral vasculature or other conditions associated with the metabolic environment. Conditions of the lung such as bronchitis, asthma and COPD may be treated. Conditions of the bowel such as inflammatory bowel disease, ulcerative colitis, Crohn's disease and pouchitis may be treated as well as neurological inflammatory conditions including muscular dystrophies. Arthritis and other joint disorders can be treated. Other human subjects include athletes to enhance endurance or sprint capacity. The reduction of inflammatory and pro-inflammatory cytokines and markers over time is a particularly useful therapeutic outcome through use of the device.

The medical device of the present disclosure is largely non-invasive in the sense that the occlusion is induced by releasable circumferential pressure. However, the monitoring device may include a fluid testing component which may require a fluid sample being taken such as to determine blood glucose levels cytokine levels as well as levels of other biochemical markers. Particular cytokines are inflammatory and pro-inflammatory cytokines such as IL-6.

Another aspect enabled herein is a therapeutic ischemic and reperfusion device with associated monitoring system comprising:

(i) non-invasive means to occlude fluid flow through a lumen and cause a localized ischemia;

(ii) means to release the occlusion to enable fluid flow through the lumen;

(iii) means to monitor vascular function at the location of the occlusion and/or remotely from the occlusion;

wherein, in use, the device induces enhanced vascular endothelial and/or metabolic function in a subject.

Particularly, the disclosure teaches a therapeutic ischemic and reperfusion device with associated monitoring system comprising:

(i) an occlusion member capable of non-invasively applying releasable circumferential pressure around an extremity of a subject's body to occlude blood flow through a blood vessel thereby inducing an ischemic condition;

(ii) a monitoring device to detect parameters of a metabolic environment within the subject including cardiovascular function or lung function at the site of the occlusion or distal or remote thereto;

wherein in use, the device therapeutically results in a reduction in inflammatory and pro-inflammatory cytokines or markers, reduced incidence or diabetes, reduced glucose intolerance, enhanced insulin sensitivity and treatment of obesity over time.

Enabled herein is a method for the treatment or prophylaxis of a metabolic condition in a subject selected from diabetes and obesity, the method comprising occluding luminal fluid flow in a vessel in the subject by the sequential application of circumferential pressure to a limb or torso on the subject containing the vessel for a time and under conditions sufficient to induce an ischemic condition, releasing the occlusion to induce reperfusion wherein the sequential ischemia and reperfusion results in a decrease in the levels of insulin compared to a subject not treated.

Another aspect enabled herein is a method for the treatment or prophylaxis of a subject with levels of IL-6, IL-10 and/or TNFα which exacerbates a disease condition, the method comprising occluding luminal fluid flow in a vessel in the subject by the sequential application of circumferential pressure to a limb or torso on the subject containing the vessel for time and under conditions sufficient to induce an ischemic condition, releasing the occlusion to induce reperfusion wherein the sequential ischemia and reperfusion results in modulation of the levels of IL-6, IL-10 and/or TNFα.

According to this embodiment, the subject may also have levels of one or more of adiponectin, myeloperoxidase and/or matrix metalloproteinase which exacerbates a disease condition.

Still another aspect taught therein is a method for the treatment or prophylaxis of a subject exposed to or who may be exposed to environmentally-induced oxidative stress, the method comprising occluding luminal fluid flow in a vessel in the subject by the sequential application of circumferential pressure to a limb or torso on the subject containing the vessel for a time and under conditions sufficient to induce an ischemic condition, releasing the occlusion to induce reperfusion wherein the sequential ischemia and reperfusion results in amelioration of the oxidative stress levels. Particular parameters include one or more of insulin, IL-6, IL-10, TNFα, adiponectin, myeloperoxidase and/or matrix metalloproteinase.

Even yet another aspect disclosed herein is a method for the treatment or prophylaxis of a disease or condition in a subject, the method comprising occluding luminal fluid flow in a vessel in the subject by the sequential application of circumferential pressure to a limb or torso on the subject containing the vessel, releasing the occlusion to include reperfusion wherein the sequential ischemia and reperfusion ameliorates symptoms of the disease or condition wherein the pressure applied is selected from the list consisting of:

(i) from the minimum pressure required to substantially occlude fluid flow through a luminal vessel to the systolic pressure or above for the treatment of Type II diabetes, obesity and selected inflammatory conditions; and

(ii) from the minimum pressure required to substantially occlude fluid flow through a luminal vessel up to but not including the systolic pressure for all other metabolic conditions.

As indicated above, selected inflammatory conditions include inflammatory conditions of the respiratory system such as bronchitis, asthma and COPD, inflammatory conditions of the bowel such as inflammatory bowel disease, ulcerative colitis, Crohn's disease, and pouchitis, inflammatory conditions of the neurological system such as muscular dystrophies and inflammatory conditions of the joints.

Physical parameters include detection of fluid flow, determination of pulse rate, determination of heart beat rate, determination of changes in temperature, determination of responses by capillaries, an assessment of flow mediated dilatation, measurements of blood flow and pulse wave velocity, pulse wave analysis, bio-impedance analysis, heart rate variability and measurement of 24 hour blood pressure as well as parameters which measure lung function such as using standard spirometry pulmonary function techniques.

Biochemical parameters include detection of an anti-inflammatory, inflammatory and/or pro-inflammatory cytokine, detection of a platelet aggregation factor, detection of oxygen levels, detection of carbon dioxide levels and detection of hemoglobin, lactate, pH, ATP, ADP, AMP, adenosine, redox voltage, erythropoietin and bradykinin levels as well as insulin, IL-6, IL-10, TNFα, adiponectin, myeloperoxidase and/or matrix metalloproteinase.

Monitoring may be at the site of the occlusion or remote from the site of the occlusion.

Also enabled herein is a method for therapeutically inducing ischemic and reperfusion conditioning in a subject, the method comprising occluding luminal fluid flow in an extremity on the subject by the application of circumferential pressure for a time and under conditions to induce an ischemic condition, releasing the occlusion and monitoring a metabolic environment within the subject wherein the ischemic conditioning results in improved lumen function over time.

In an embodiment, contemplated herein is a method for therapeutically inducing ischemic and reperfusion conditioning in a subject, the method comprising occluding blood vessel fluid flow in an appendage on the subject by the application of circumferential pressure for a time and under conditions to induce an ischemic condition, releasing the occlusion and monitoring a metabolic environment within the subject wherein the ischemic conditioning results in improved blood vessel function, metabolic function, cardiovascular function and/or lung function over time.

As indicated above, a convenient means for occluding fluid flow is via use of a pressure cuff or tourniquet.

An embodiment contemplated herein is a method for the treatment or prophylaxis of metabolic condition in a subject selected from diabetes, obesity and an inflammatory response, said method comprising applying an inflatable and deflatable pressure cuff or tourniquet adapted to fit around a limb or portion of a torso of the subject wherein upon inflation of the pressure cuff or tightening of the tourniquet, fluid flow through a luminal vessel in the limb or torso is occluded causing localized ischemia and, when deflated, enables fluid to flow through the luminal vessel and subjecting the pressure cuff or tourniquet to multiple cycles of inflation and deflation or tightening and releasing operated by a controller operably connected to the pressure cuff or tourniquet wherein the pressure applied is between a selected pressure, the multiple cycles of inflation and deflation or tightening and releasing being for a time and under conditions for an efficacious vascular response compared to a subject which has not undergone the treatment.

As above, the selected pressure is selected from:

(i) the minimum pressure required to substantially occlude fluid flow through a luminal vessel up to systolic pressure or above; and

(ii) the minimum pressure required to substantially occlude fluid flow through a luminal vessel up to but not including the systolic pressure.

By “monitoring” the “metabolic environment” includes assessing vascular function or assessing any of the physical and/or biochemical parameters as listed above.

The time of occlusion may be from 10 seconds to 6 minutes such as 10, 20, 30, 40, 50, 60, 70, 80, 90, 100, 110, 120, 130, 140, 150, 160, 170 or 180 seconds or from 1 minute to 50 minutes such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 minutes. Generally, the time of occlusion is between from 10 seconds to 15 minutes. Times of from 3 to 6 minute and 3 to 5 minute intervals are taught herein. The present disclosure proposes a cyclical occlusion and reperfusion such as every minute, hour, day or week and from 1 to 20 times per minute, hour, day or week such as 1, 2, 3, 4. 5, 6, 7, 8, 9, 10, 11, 12, 13, 14, 15, 16, 17, 18, 19 or 20 times per minute, hour, day or week.

Another aspect enabled herein is the use of an occluding member adapted to releaseably restrict luminal fluid flow following circumferential pressure of an appendage in which the lumen is located and an associated monitoring element in the manufacture of an ischemic conditioning device.

Furthermore, taught herein is a use of an inflatable and deflatable pressure cuff or tourniquet adapted to fit around a limb or portion of a torso of a subject wherein upon inflation of the pressure cuff or tightening of the tourniquet, fluid flow through luminal vessel in the limb or torso is occluded causing localized ischemia and, when deflated, enables the fluid to flow through the luminal vessel, in the manufacture of an ischemic and reperfusion device to induce an efficacious vascular response in a subject.

As indicated above, an efficacious vascular response includes improvement of HOMA-IR as well as levels of one or more of insulin, IL-6, IL-10, TNFα, adiponectin, myeloperoxidase and/or matrix metalloproteinase.

Furthermore, a further aspect is the use of an inflatable and deflatable pressure cuff or tourniquet adapted to fit around a limb or portion of a torso of a subject wherein upon inflation of the pressure cuff or tightening of the tourniquet, fluid flow through a luminal vessel in the limb or torso is occluded causing localized ischemia and, when deflated, enables the fluid to flow through the luminal vessel;
a controller operably connected to the pressure cuff or tourniquet configured to inflate the cuff or tighten the tourniquet to a selected pressure and to deflate the cuff or release the tourniquet; and
a monitor configured to monitor luminal vessel and/or metabolic function at the site of the occlusion or remote to the site of occlusion in the manufacture of an ischemic and reperfusion device to induce an efficacious vascular response in a subject.

The present disclosure further provides a method of enhancing a subject's metabolic environment, the method comprising cyclically releaseably occluding blood flow through a vessel for a time and under conditions to induce remote ischemic conditioning and improved metabolic function including cardiovascular function.

The present disclosure teaches the use of an occluding member adapted to releaseably restrict blood vessel following circumferential pressure of an appendage in which the blood vessel is located and an associated monitoring element in the manufacture of an ischemic conditioning device.

The monitoring member may also include an algorithm-based or computer-based component to input, store or manipulate data on biochemical and/or physical parameters alone or in combination with information such as sex, age, general health characteristics and the like of the subject. Generally, input data are collected based on information gathered by the monitoring member and subjected to an algorithm or computer program to assess the statistical significance of any elevation or reduction in levels of parameters which information is then output data. Computer software and hardware for assessing input data and output data are encompassed by the present disclosure.

Another aspect taught herein is a method of enhancing a subject's metabolic environment, the method comprising subjecting a recipient to remote ischemic conditioning and determining physical and/or biochemical parameters by a monitoring member associated with an occlusion member and then subjecting the parameters to multivariate or univariate analysis to determine whether the metabolic environment has been enhanced relative to a control or standardized norms. A control includes a subject not having undergone ischemic and reperfusion therapy.

The device herein permits integration into existing or newly developed pathology architecture or platform systems. For example, a method is contemplated allowing a user to determine the status of a subject with respect to its metabolic environment, the method including:

(a) receiving data in the form of physical and/or biochemical parameters prior to or following remote ischemic conditioning from a user via a communication network;

(b) processing the subject data via an algorithm which provides a metabolic index value;

(c) determining the status of the subject in accordance with the results of the metabolic index value in comparison with predetermined values; and

(d) transferring an indication of the status of the subject to the user via the communications network.

The user may be the subject or a carer including a physician, clinician or veterinarian.

Conveniently, the method generally further includes:

(a) having the user determine the data using a remote end station; and

(b) transferring the data from the end station to the base station via the communications network.

The base station can include first and second processing systems, in which case the method can include:

(a) transferring the data to the first processing system;

(b) transferring the data to the second processing system; and

(c) causing the first processing system to perform the algorithmic function to generate the disease index value.

The method may also include:

(a) transferring the results of the algorithmic function to the first processing system; and

(b) causing the first processing system to determine the status of the subject.

In this case, the method also includes at lest one of:

(a) transferring the data between the communications network and the first processing system through a first firewall; and

(b) transferring the data between the first and the second processing systems through a second firewall.

The second processing system may be coupled to a database adapted to store predetermined data and/or the algorithm, the method include:

(a) querying the database to obtain at least selected predetermined data or access to the algorithm from the database; and

(b) comparing the selected predetermined data to the subject data or generating a predicted probability index.

The second processing system can be coupled to a database, the method including storing the data in the database.

The method can also include causing the base station to:

(a) determine payment information, the payment information representing the provision of payment by the user; and

(b) perform the comparison in response to the determination of the payment information.

Also contemplated herein is a base station for determining the status of a subject with respect to its metabolic environment, the base station including:

(a) a store method;

(b) a processing system, the processing system being adapted to;
(i) receive subject data from a user via a communications network, the data including levels of physical and/or biochemical parameters prior to or following remote ischemic conditioning from a subject;
(ii) performing an algorithmic function including comparing the data to predetermined data;
(iii) determining the status of the subject in accordance with the results of the algorithmic function including the comparison; and

(c) output an indication of the status of the subject to the user via the communications network.

The processing system can be adapted to receive data from a remote end station adapted to determine the data.

The processing system may include:

(a) a first processing system adapted to:
(i) receive the data; and
(ii) determine the status of the subject in accordance with the results of the algorithmic function including comparing the data; and

(b) a second processing system adapted to:
(i) receive the data from the processing system;
(ii) perform the algorithmic function including the comparison; and
(iii) transfer the results to the first processing system.

The base station typically includes:

(a) a first firewall for coupling the first processing system to the communications network; and

(b) a second firewall for coupling the first and the second processing systems.

The processing system can be coupled to a database, the processing system being adapted to store the data in the database.

Reference to an “algorithm” or “algorithmic function” as outlined above includes the performance of an univariate or multivariate analysis function. A range of different architectures and platforms may be implemented in addition to those described above. It will be appreciated that any form of architecture suitable for implementing the present disclosure may be used. However, one beneficial technique is the use of distributed architectures.

It will also be appreciated that in one example, the end stations can be hand-held devices, such as PDAs, mobile phones, or the like, which are capable of transferring the subject data to the base station via a communications network such as the Internet, and receiving the reports.

In the above aspects, the term “data” means the levels or concentrations of the biomarkers. The “communications network” includes the internet. When a server is used, it is generally a client server or more particularly a simple object application protocol (SOAP).

Hence, a business model is provided in which a subject or carer may apply remote ischemic conditioning and have the data monitored by the subject or carer or via a communication network to a remote user. An aspect enabled herein is a method for the treatment or prophylaxis of a metabolic condition in a subject selected from diabetes and obesity, said method comprising occluding luminal fluid flow in a vessel in the subject by the sequential application of circumferential pressure to a limb or torso on the subject containing the vessel for a time and under conditions sufficient to induce an ischemic condition, releasing the occlusion to induce reperfusion wherein the sequential ischemic and reperfusion results in a decrease in the fluid levels of insulin compared to a subject not treated.

Aspects enabled herein are further taught by the following non-limiting Examples.

### Example 1

### Demonstration of rIPC Treatment on Healthy Individuals Vascular, Glucose, Insulin and Inflammation Response

Data are obtained on the vascular protective effects against a high fat, high glucose meal following a period of treatment with rIPC. In this study, normal healthy adult male participants (n=18) underwent assessment of vascular function using the EndoPAT 2000 system (Itamar). This non-invasive system employs probes placed on the fingertips to measure the response of the capillaries in the fingertips to a brief (5 minute) period of ischemia and subsequent reperfusion, similar to the protocol employed in the assessment flow mediated dilatation (FMD) of the brachial artery, to derive the index reactive hyperaemic index (RHI). Following baseline assessment, subjects ate a high-fat, high-glucose meal and had repeated assessments of vascular function at 1 and 2 hours post-meal. Subjects subsequently underwent daily rIPC treatment using the same protocol of 4 cycles of 5 minutes of ischemia followed by 5 minutes of reperfusion for 7 days. On the eighth day, subjects underwent the same vascular assessment at baseline and following a meal of the same constituents. Following baseline assessment, subjects ate a high-fat, high-glucose meal and had repeated assessments of vascular function at 1 and 2 hours post-meal.

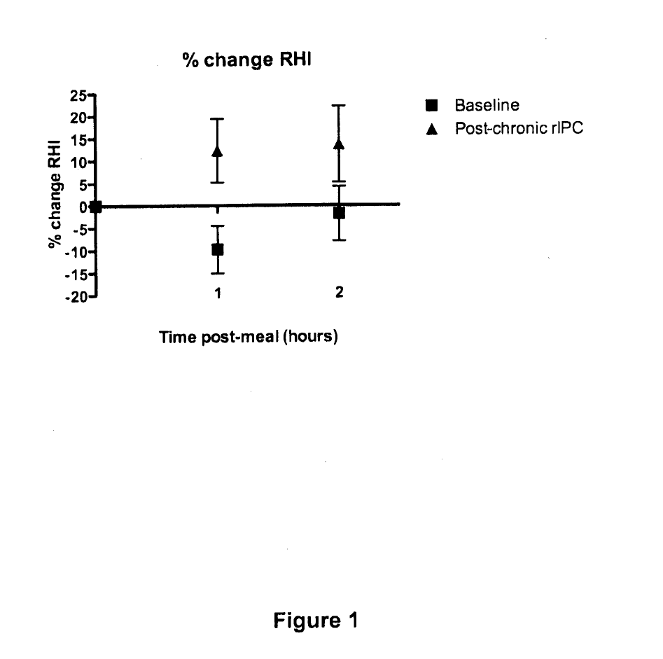

Following a period of treatment with daily rIPC, the vascular dysfunction induced by the high-fat and glucose meal was significantly less as demonstrated by a significant change (p<0.003) in the reactive hyperemic index (RHI) as a percentage of pre-meal values (time 0) and at 1 and 2 hours after a high-fat, high-glucose meal (FIG. 1). These data demonstrate that endothelial dysfunction induced by ingestion of a high-fat, high-glucose meal is modified in individuals following a period of chronic rIPC.

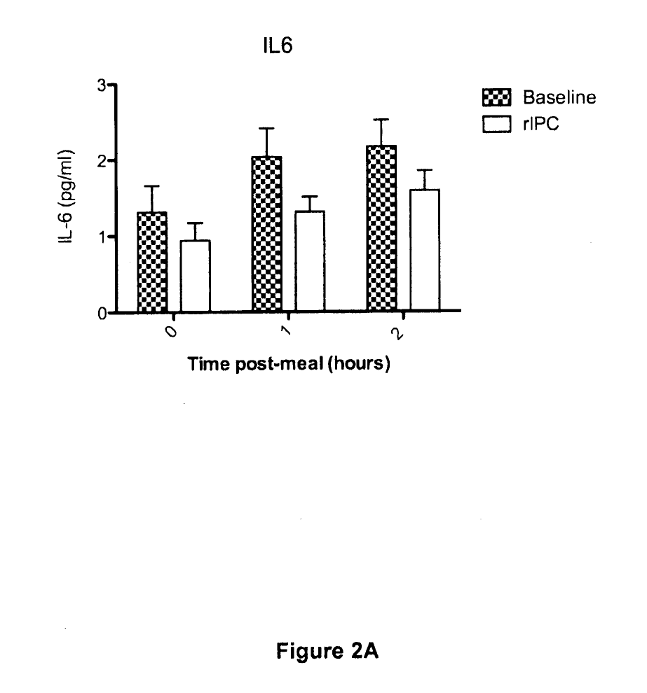

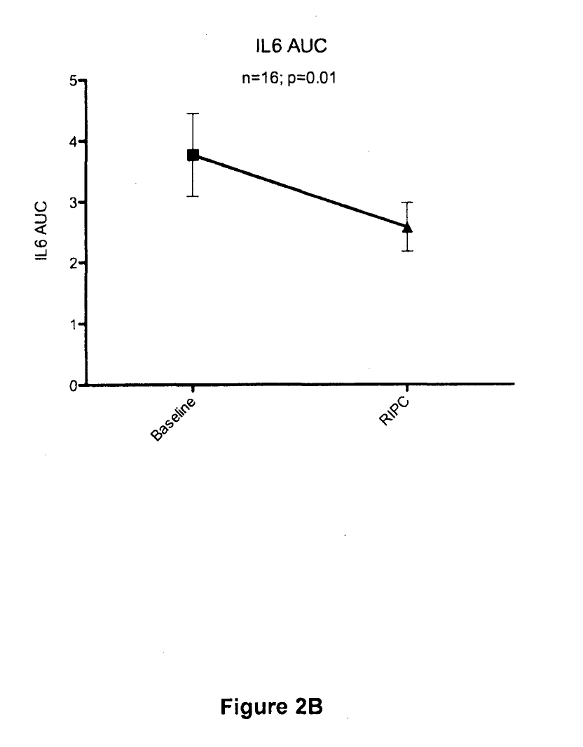

The levels of levels of the cytokine, IL-6 in subjects (n=16) receiving treatment with rIPC vs controls (baseline) at pre-meal (time 0) and then at 1 and 2 hours after eating a high fat, high glucose meal (FIG. 2A). There was a significant reduction (AUC; p=0.01) in the serum levels of IL-6 in response to the fatty meal (n=16) following a week of daily rIPC treatment (FIG. 2B), further suggesting modification of the inflammatory response. These are data from seven individuals. The data represent the response to a brief exposure of the vascular endothelium to the insult of high blood levels of glucose and triglycerides, which elicits a low grade inflammatory response. The situation in obese subjects and diabetics is altered with much more prolonged metabolic derangement and also more persistent activation of the inflammatory milieu even in the absence of high glucose and fat in the bloodstream. Inflammation similarly persistently activated to a greater degree in patients with conditions such as connective disease.

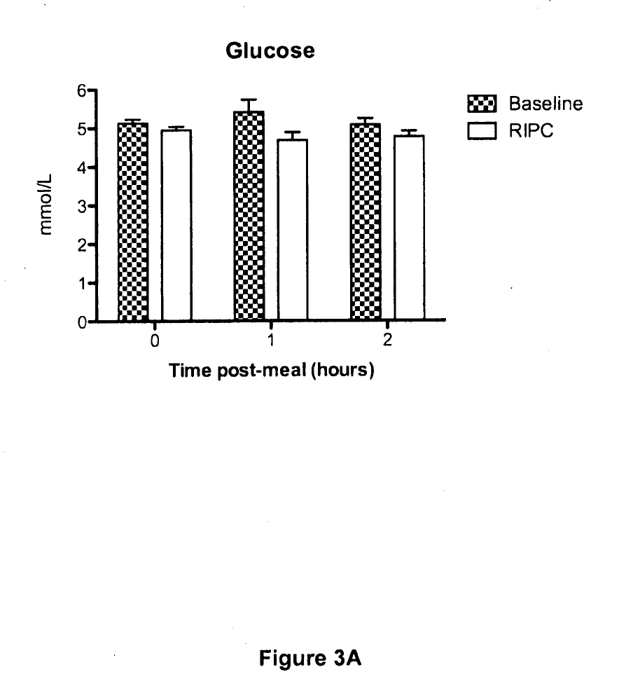

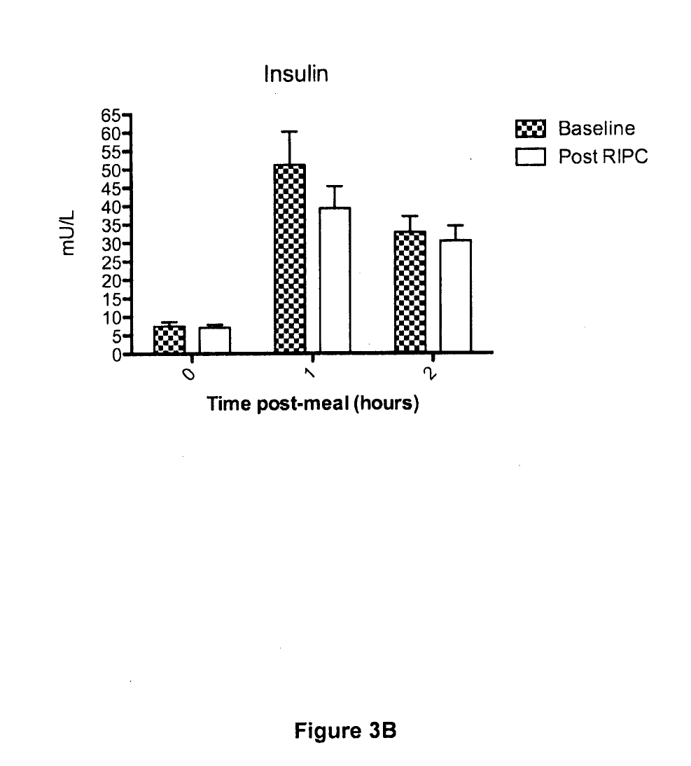

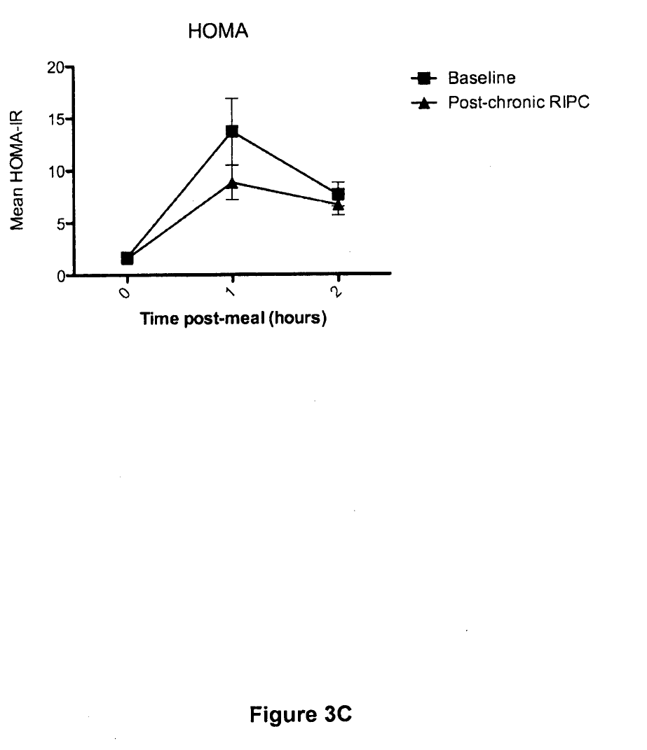

In addition to these change's in the inflammatory profile, important changes in glucose homeostasis were observed in this pilot study (FIG. 3A-C). In these normal healthy males, glucose levels were well controlled after the high-fat, high-glucose meal, however there was a lower area under the curve for HOMA-IR after the period of rIPC. This greater insulin sensitivity is of particular interest in the conditions of obesity and type 2 diabetes. The controlled serum glucose levels in normal healthy males (n=18) alter ingestion of a high-fat, high-glucose meal both at baseline and alter a week of daily rIPC is shown in FIG. 3A. Lower levels of insulin (FIG. 3B) were released alter the week of rIPC indicting improved sensitivity as shown by the lower area under the curve (AUC) for homeostatic model assessment for insulin resistance (HOMA-IR) IIOMA-IR (FIG. 3C) after consumption of the standard high fat and high glucose meal at baseline prior to and following a week of daily preconditioning by inflating the cuff on the upper arm greater than systolic blood pressure for 3 cycles of 5 minutes of ischaemia followed by cull release and 5 minutes of reperfusion.

### Example 2

### Demonstration of rIPC Treatment (Lower than Systolic Pressure) on Healthy Individuals and their Vascular, Glucose and Insulin Response

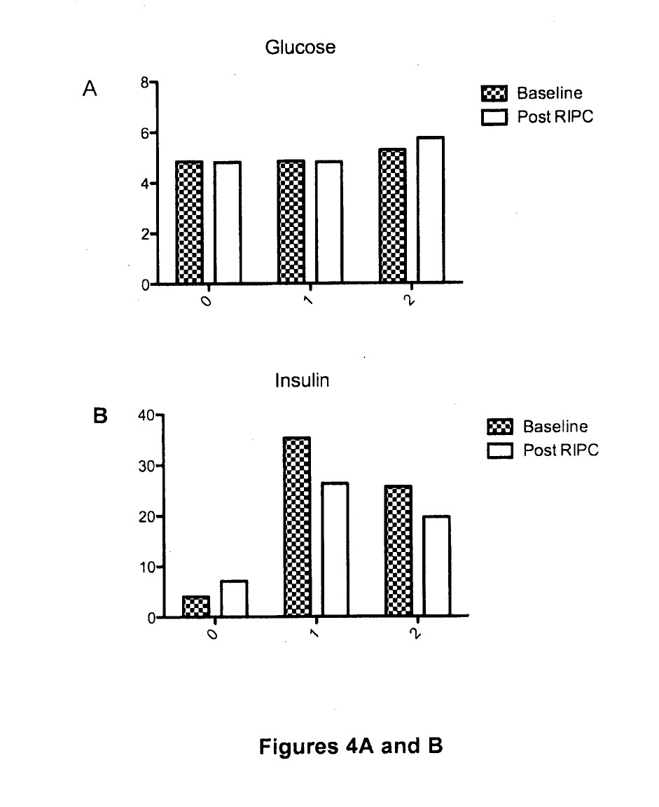

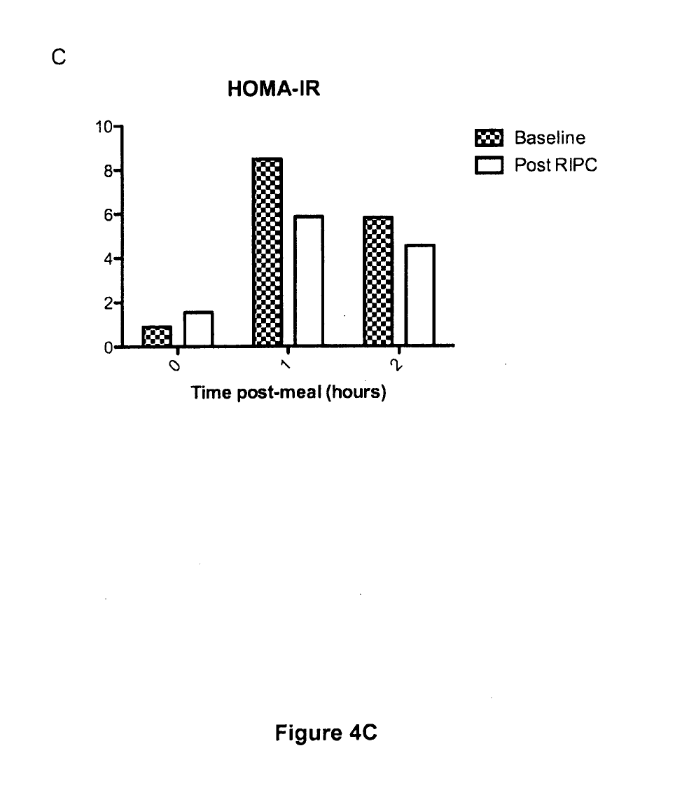

The insulin and glucose homeostatic response after consumption of the standard high fat and high glucose meal after preconditioning by inflating the cuff on the upper arm to 20 mmHg greater than diastolic blood pressure and less than systolic pressure are shown in FIG. 4A-C (n=1). Besides the pressure to which the cuff was inflated, the same daily protocol of 3 cycles of 5-minutes occlusion and 5 minutes of reperfusion for one week was followed. A similar reduction IIOMA-IR AUC is observed when compared to the mean response of the group of 18 normals (refer to Example 1) undergoing the standard protocol of exceeding systolic blood pressure for occlusion.

### Example 3

### Use of Myeloperoxidase as a Marker of Oxidative Stress

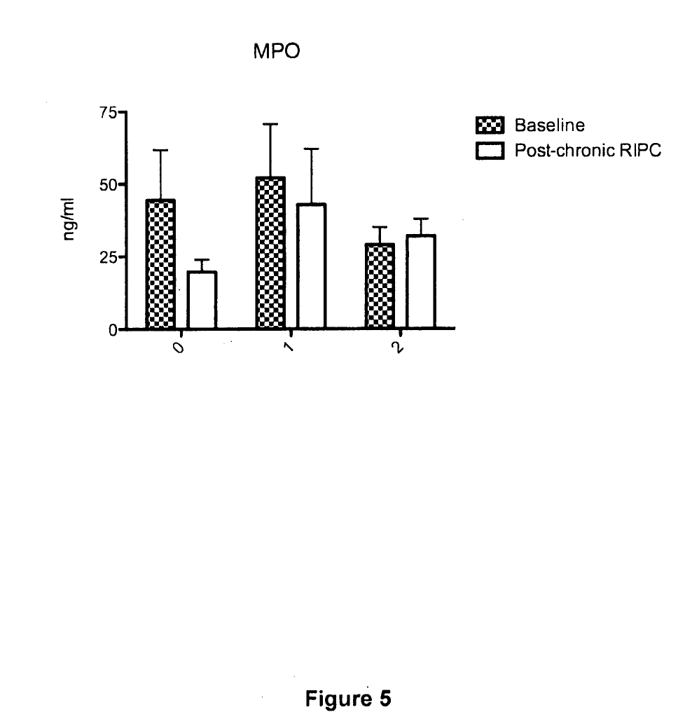

Dietary induced oxidative stress is widely recognized due to the Western diet. The body is subjected to this environmental stress each time unhealthy (high sugar and/or high fat) meals are consumed. FIG. 5 shows the levels of myeloperoxidase (MPO) in the body in response to injury or danger. Subjecting a patient to rIPC cause some modification of the individual's release of MPG with lower levels in the resting state and also less evidence of oxidative stress following the high fat and high glucose meal.

### Example 4

### Development of a Remote Ischemic Conditioning Device

The device designed to deliver the rIPC protocol of repeated 4 cycles of 5 minutes of ischemic followed by reperfusion for 5 minutes. The device has the ability to be programmed to perform the protocol of occlusion of arterial blood flow to the limb using a small inflatable cuff. Importantly the device has an associated monitoring function component to assess vascular function. The monitoring component utilises either finger pulse plethysmography or strain gauge plethysmography to measure forearm blood flow as a measure of vascular endothelial function. The prototype research device is flexibility to change parameters and also to record and upload all the monitoring data for analysis. The latter is useful for development of ranges and targets for individuals with different disease conditions.

### Example 5

### Evaluation of the Remote Ischemic Conditioning Device

In order to test the device, the effects of chronic rIPC are observed. The device is used in subjects with low grade vasculitis associated with connective tissue disease. Patients with systemic lupuserythematosus, systemic juvenile rheumatoid arthritis and dermatomyositis are studied. Even when in remission i.e. not in an active phase of disease these patients are well known to have serum biomarkers of increased vascular inflammation (Chen et al. Ann. Rheum. Dis. 61(2):167-170, 2002).

### Study and Subjects

Study 1. Study of patients with connective tissue disease (n=10) using standard hospital equipment (daily rIPC for 10 days).

Study 2. Study of patients with connective disease guided by pilot data from study 1, using standard hospital equipment (daily rIPC for 10 days).

Study 3. Study of normal healthy volunteers using prototype device (n=10), (daily rIPC for 10 days).

Study 4. Study of patients with connective tissue disease using prototype device (n=10), (daily rIPC for 10 days).

Study 5. Rat model of type 2 diabetes (n=20).

Study 6. Human model of Type 2 diabetes and obesity using above systolic endpoint for rIPC.

Study 7 Human model of Type 2 diabetes and obesity using above diastolic endpoint and less that systolic endpoint for rIPC.

Study 8 Human model of lung disease.

Study 9 Human model of inflammatory bowel disease.

Human Studies of Patients with Vasculitis.
Subjects Act as their Own Controls.

### Baseline Measurements

1. Height weight and waist circumference. 2. Blood pressure. 3. Baseline fasting blood tests. A 10 ml blood sample is taken in serum gel tube, centrifuged at 3000 rpm for 10 minutes and the serum frozen at −70° C. These samples are examined for levels IL-6, IL-10, IFN-γ, TNFα, cellular adhesion molecules (sICAM1 and sVCAM1) and F-select in using a multiplex assay (Luminex [Registered Trade Mark]). 4. Vascular function measurements. These are performed in the supine position after a 10 minute period of acclimatisation. Subjects will be fasted for at least 6 hours prior to examination. Flow mediated dilatation using finger plethysmography (EndoPAT) are performed.

### Microvascular Function.

The EndoPAT system is employed to assess microvascular function. Non-invasive probes is placed on the tips of the middle finger of each hand. After a 10 min equilibration period, baseline measurements of fingertip blood flow are made for a period of 5 minutes. Forearm blood flow to the right hand will be occluded by inflating a blood pressure cuff to 50 mmHg above systolic pressure for 5 minutes. Following release of the cuff, the change in fingertip blood flow is reassessed. The percentage change in blood flow following the period of ischemia is measured using proprietary software.

### Preconditioning Protocol.

The subjects undergo daily pre-conditioning, for 10 days using the same protocol of 3 cycles of 5 minutes of limb ischemia and subsequent reperfusion.

### Follow-Up Assessment.

Following the 10 days of pre-conditioning, subjects undergo the same measurements as at baseline with blood tests and non-invasive vascular function assessment.

### Statistical Analysis.

The initial study of 10 subjects provides data with which to guide subsequent studies.

Prototype device (Studies 3 and 4).

In Study 3, a preliminary safety assessment in normal individuals is performed with the prototype device (daily rIPC for 10 days). In study 4, 10 subjects with vasculitis are studied to compare safety and effectiveness of the prototype relative to the standard hospital equipment. Besides the use of the prototype device, the same protocol as in studies 1 and 2 is employed, with baseline fasting blood sampling and vascular function assessment, followed by 10 days of daily rIPC and then reassessment with blood tests and vascular assessment. In addition to a further safety assessment, this provides preliminary data as to the efficacy of the device in comparison with the response using the standard hospital equipment.

### Example 6

### Rat Model of Type 2 Diabetes

The feasibility of daily rIPC is demonstrated in two rat models of obesity (cafeteria diet induced and Zucker obese fatty rats). The effects of repeated rIPC in the Zucker Diabetic Fatty rat model of Type 2 diabetes is studied. Sham treatment and rIPC is carried out in a Perspex restraining tube. Adult ZDF rats (n=24) aged 8 months are randomized to one of four groups, 1. Short course controls; placed in Perspex restraining tube daily for 1 week, but no tail artery occlusion. 2. Short course rIPC; daily rIPC by occlusion of the tail artery using a vascular occluder for 5 minutes followed by reperfusion for 5 minutes (4 cycles) for 1 week. 3. Prolonged course controls; placed in Perspex restraining tube 3 times/week for 4 weeks, but no tail artery occlusion. 4. Prolonged course rIPC; rIPC are carried out 3 times/week for 4 weeks. The rationale for this different frequency of rIPC is based on the well reported duration of effect of a single rIPC stimulus which has a second window with onset approximately 24 hours later and lasting for approximately 72 hours. It is also an important pragmatic issue to determine whether less frequent rIPC treatment is effective.

During the 4 week period, animals have weekly blood sampling for fasting glucose and insulin levels. At the end of this period, animals are euthanized and organs (brain, liver, heart, kidneys, skeletal muscle and abdominal fat) frozen in liquid nitrogen. Tissues and blood are examined for alteration of oxidative stress markers and inflammation. In addition to serum levels of inflammatory cytokines, adipose tissue are examined for levels of TNFα, IL-6 and adiponectin RNA, and skeletal muscle are examined for levels of PPARγ and AMP kinase.

### Example 7

### Human Model of Obesity and Type 2 Diabetes

Subjects Act as their Own Controls

### Baseline Measurements

1. Height weight and waist circumference; 2. Blood pressure 3. Baseline fasting blood tests. A 10 ml blood sample will be taken in serum gel tube, centrifuged at 3000 rpm for 10 minutes and the serum frozen at −70° C. These samples are examined for levels of glucose, insulin and HbA1C, IL-6, IL-10, IFN-γ, TNFα, cellular adhesion molecules (sICAM1 and sVCAM1) and E-select in using a multiplex assay (Luminex [Registered Trade Mark]). 4. Vascular function measurements. These are performed in the supine position after a 10 minute period of acclimatisation. Subjects will be fasted for at least 6 hours prior to examination. Flow mediated dilatation using finger plethysmography (EndoPAT) are performed.

### Microvascular Function.

The EndoPAT system is employed to assess microvascular function. Non-invasive probes is placed on the tips of the middle finger of each hand. After a 10 min equilibration period, baseline measurements of fingertip blood flow will be made for a period of 5 minutes. Forearm blood flow to the right hand will be occluded by inflating a blood pressure cuff to 50 mmHg above systolic pressure for 5 minutes (or alternative method refer to Study 7 in Example 8 below). Following release of the cuff, the change in fingertip blood flow is reassessed. The percentage change in blood flow following the period of ischemia is measured using proprietary software.

### Preconditioning Protocol.

The subjects undergo daily pre-conditioning, for 10 days using the same protocol of 3 cycles of 5 minutes of limb ischemia and subsequent reperfusion.

### Follow-Up Assessment.

Following the 10 days of pre-conditioning, subjects undergo the same measurements as at baseline with blood tests and non-invasive vascular function assessment.

### Statistical Analysis.

The initial study of 10 subjects provides data with which to guide subsequent studies.

### Example 8

### Diastolic Occlusion in a Human Model of Obesity and Type 2 Diabetes

Subjects Act as their Own Controls.

### Baseline Measurements

1. Height weight and waist circumference; 2. Blood pressure 3. Baseline fasting blood tests. A 10 ml blood sample will be taken in serum gel tube, centrifuged at 3000 rpm for 10 minutes and the serum frozen at −70° C. These samples are examined for levels of glucose, insulin and HbA1C, IL-6, IL-10, IFN-γ, TNFα, cellular adhesion molecules (sICAM1 and sVCAM1) and E-select in using a multiplex assay (Luminex [Registered Trade Mark]) 4. Vascular function measurements. These are performed in the supine position after a 10 minute period of acclimatisation. Subjects will be fasted for at least 6 hours prior to examination. Flow mediated dilatation using finger plethysmography (EndoPAT) are performed.

### Microvascular Function.

The EndoPAT system is employed to assess microvascular function. Non-invasive probes is placed on the tips of the middle finger of each hand. After a 10 min equilibration period, baseline measurements of fingertip blood flow will be made for a period of 5 minutes. Forearm blood flow to the right hand will be occluded by inflating a blood pressure cuff to 50 mmHg above systolic pressure for 5 minutes. Following release of the cuff, the change in fingertip blood flow is reassessed. The percentage change in blood flow following the period of ischemia is measured using proprietary software.

### Diastolic Preconditioning Protocol.

The subjects undergo daily pre-conditioning by inflating a cuff on the upper limb to 20 mmHg above diastolic pressure, for 10 days using the same protocol of 3 cycles of 5 minutes of limb ischemia and subsequent reperfusion.

### Follow-Up Assessment.

Following the 10 days of pre-conditioning, subjects undergo the same measurements as at baseline with blood tests and non-invasive vascular function assessment.

### Statistical Analysis.

The initial study of 20 subjects in each group (obese n=20, type 2 diabetics n=20) and provides data with which to guide subsequent studies.

### Example 9

### Human Model of Inflammatory Lung Diseases

Subjects Act as their Own Controls.

Patients with common lung conditions with an inflammatory component such as asthma and chronic bronchitis are studied.

### Baseline Measurements

Subjects are fasted for at least 6 hours prior to examination.

1. Height weight and waist circumference;
2. Blood pressure
3. Baseline fasting blood tests. A 10 ml blood sample is taken in serum gel tube, centrifuged at 3000 rpm for 10 minutes and the serum frozen at −70° C. These samples are examined for levels of IL-6, IL-10, IFN-γ, and TNFα, using a multiplex assay (Luminex [Registered Trade Mark]).
4. Lung function measurements using standard spirometry and pulmonary function techniques. Measurement of distal pulmonary inflammation is also be performed.

The subjects undergo regular chronic pre-conditioning by inflating a cuff on the upper limb, using the same protocol of 3 cycles of 5 minutes of limb ischemia and subsequent reperfusion.

### Follow-Up Assessment

Following the period of chronic repeated pre-conditioning, subjects undergo the same measurements as at baseline with blood tests, pulmonary function assessment and measurement of distal pulmonary inflammation.

### Example 10

### Human Model of Inflammatory Bowel Disease

Subjects Act as their Own Controls.

Patients with chronic bowel conditions with an inflammatory component such as ulcerative colitis and Crohn's disease are studied.

### Baseline Measurements

Subjects are fasted for at least 6 hours prior to examination.

1. Height weight and waist circumference;
2. Blood pressure
3. Baseline fasting blood tests. A 10 ml blood sample will be taken in scrum gel tube, centrifuged at 3000 rpm for 10 minutes and the serum frozen at −70° C. These samples are examined for levels of IL-6, IL-10, IFN-γ, TNFα using a multiplex assay (Luminex [Registered Trade Mark]).
4. Stool specimens are examined for indicators of bowel inflammation.

The subjects undergo repeated chronic pre-conditioning by inflating a cuff on the upper limb, using the same protocol of 3 cycles of 5 minutes of limb ischemia and subsequent reperfusion.

### Follow-Up Assessment.

Following the period of regular chronic pre-conditioning, subjects undergo the same measurements as at base line with blood and stool tests.

Those skilled in the art will appreciate that aspects disclosed here are susceptible to variations and modifications other than those specifically described. It is to be understood that these aspects include all such variations and modifications. The disclosure also contemplates all of the steps, features, compositions and compounds referred to or indicated in this specification, individually or collectively, and any and all combinations of any two or more of said steps or features.

### BIBLIOGRAPHY

Chen et al, Ann. Rheum. Dis. 61(2):167-170, 2002
Cheung et al, J. Am. Coll. Cardiol. 47:2277-2282, 2006
Das and Maulik, Cardiovascular Research 70:254-263, 2006
Kharbanda et al, Circulation 103(12):1624-1630, 2001
Kharbanda et al, Heart 92(10):1506-1511, 2006
Konstantinov ey al. Physiol. Genomics. 19:143-150, 2004
Konstantinov et al, J. Thorac. Cadiovasc. Surg. 130(5):1326-1332, 2005
Laskey and Beach, J. Am. Coll. Cardiol. 42:998-1003, 2003
Lessar et al, J. Am. coll. Cardiol. 42:175-180, 2003
Linden et al, J. Throm. Haem. 4:2670-2677, 2006
Lindhardt et al, Heart 90:425-430, 2004
Matthews et al, Diahetologia 28(7):412-419, 1985
Murray et al, Circulation 74:1124-1136, 1986
Przyklenk et al, Circulation 87:893-899, 1993
Shimizu et al, J. Surg. Res, 158(1):155-161, 2010
Sullivan et al, Brit. J. Surg. 96:381-390, 2009
Teoh et al, Cardiovasc. Res. 53:175-180, 2002
Weber, Nature Medicine 16:760-762, 2010

1.-36. (canceled)

37. A method for treating obesity in a subject in need thereof, said method comprising occluding luminal fluid flow in a vessel in the subject by the application of circumferential pressure to a limb or torso on the subject containing the vessel sufficient to induce an ischemic condition, releasing the occlusion to induce reperfusion and sequentially repeating the occlusion and reperfusion for a time and under conditions to normalize levels of adiponectin to ameliorate obesity or a condition associated therewith.

38. The method of claim 1 wherein the repeated occlusion and reperfusion further normalize levels of IL-6.

39. The method of claim 1 wherein the condition associated with obesity is obesity-induced inflammation and/or insulin resistance.

40. The method of claim 1 wherein the condition associated with obesity is cardiovascular disease.

41. The method of claim 1 wherein the condition associated with obesity is type I diabetes.

42. The method of claim 1 wherein the condition associated with obesity is type II diabetes.

43. The method of claim 1 wherein the condition associated with obesity is dietary-induced oxidative stress.

44. A method for treating a condition selected from inflammation, bowel disease, respiratory disease and an inflammatory neuropathology, in a subject in need thereof, said method comprising occluding luminal fluid flow in a vessel in the subject by the application of circumferential pressure to a limb or torso on the subject containing the vessel sufficient to induce an ischemic condition, releasing the occlusion to induce reperfusion and sequentially repeating the occlusion and reperfusion for a time and under conditions to normalize levels of adiponectin and Interleukin-6 (IL-6) to ameliorate symptoms of the condition.

45. The method of claim 8 wherein the inflammation is selected from bronchitis, asthma, COPD, Crohn's disease, inflammatory bowel disease, ulcerative colitis and a muscular dystrophy.

46. The method of claim 1, wherein the duration for each occlusion and reperfusion step is from 2 minutes to 8 minutes.

47. The method of claim 1, wherein the duration of the sequential occlusion and reperfusion treatment is for from 5 minutes to 60 minutes.

48. The method of claim 1, wherein the fluid is blood in a blood vessel.

49. The method of claim 1, wherein an inflatable and deflatable pressure cuff or tourniquet adapted to fit around a limb or portion of a torso of the subject is applied wherein upon inflation of the pressure cuff or tightening of the tourniquet, fluid flow through a luminal vessel in the limb or torso is occluded causing localized ischemia and, when deflated, enables fluid to flow through the luminal vessel and subjecting the pressure cuff or tourniquet to multiple cycles of inflation and deflation or tightening and releasing operated by a controller operably connected to the pressure cuff or tourniquet.

50. The method of claim 8, wherein the duration for each occlusion and reperfusion step is from 2 minutes to 8 minutes.

51. The method of claim 8 wherein the duration of the sequential occlusion and reperfusion treatment is for from 5 minutes to 60 minutes.

52. The method of claim 8 wherein the fluid is blood in a blood vessel.

53. The method of claim 8 wherein an inflatable and deflatable pressure cuff or tourniquet adapted to fit around a limb or portion of a torso of the subject is applied wherein upon inflation of the pressure cuff or tightening of the tourniquet, fluid flow through a luminal vessel in the limb or torso is occluded causing localized ischemia and, when deflated, enables fluid to flow through the luminal vessel and subjecting the pressure cuff or tourniquet to multiple cycles of inflation and deflation or tightening and releasing operated by a controller operably connected to the pressure cuff or tourniquet.
